(19) United States
(12) Patent Application Publication
(10) Pub. No.: US 2004/0022679 A1
(43) Pub. Date: Feb. 5, 2004

(54) Decontamination system for chemical and biological agents

(76) Inventors: Benedict B. St. Onge, San Marco, CA (US); Mirat D. Gurol, San Diego, CA (US)

Correspondence Address: Michael J. Ram, KOPPEL, JACOBS, PATRICK & HEYBL, Suite 107, 555 St. Charles Drive, Thousand Oaks, CA, 91360, US

(21) Appl. No.: 10/286,044

(22) Filed: Nov. 1, 2002

Related U.S. Application Data
(60) Provisional application No. 60/338,564, filed on Nov. 2, 2001.

(51) Int. Cl. A61L 9/015

(52) U.S. Cl. 422/62; 422/124

(57) ABSTRACT

An automated computer based system for controlling ozonation devices and the delivery, in a controlled manner, of ozone for decontaminating enclosed spaces, such as rooms and buildings, to destroy microorganisms and chemicals that contaminate the space or are used as biological or chemical warfare agents is described. Air with ozone in the gas form is used to completely fill the entire space, including the vents, to inactivate bacteria, viruses, spores, and cysts, and to break down biological and chemical toxins with minimal damage to the contents of the buildings. An algorithm for addressing the system variables is provided so that the required times for each stage of the decontamination process can be controlled and the operational variables can be set to reflect the requirements of the system variables

This application claims benefit of Provisional Application No. 60/338,564 filed Nov. 2, 2001.

The system described is an automated ozone-generating device for the effective use of ozone in destruction of chemical and biological agents in enclosed spaces. Biological or chemical contamination is both a Department of Defense and a commercial problem.

### BACKGROUND

There are currently very few technologies available for decontaminating the air and the surfaces in enclosed spaces. These potentially include technologies that use hydrogen peroxide foam, heat, and electromagnetic or UV radiation. Decontamination of a room using peroxide foam requires filling the entire cavity and covering all the surfaces with the foam mixture. Complete coverage of the surfaces is difficult, the process is slow, and cleanup after application is difficult. The effectiveness of heat on spores has not been evaluated well. Furthermore, excessive heat application might damage sensitive material and cause accidental fire. UV and electromagnetic radiation need to be directed onto microorganisms, i.e. direct contact is required. Therefore, the radiation technologies are not appropriate for cleaning of all the surfaces in a building. Furthermore, radiation will have little effect on may chemical contaminants.

Ozone gas by contrast is capable of diffusing into crevices and difficult-to-reach areas in buildings. It leaves no residue on treated surfaces. The decontaminated room requires no post treatment cleanup because ozone naturally decomposes into oxygen in a matter of hours. In addition, ozone is expected to be effective on both biological and chemical contaminants.

Ozone is a chemical that functions as a very strong oxidant and disinfectant. Ozone has been used commercially for almost 100 years to kill many types of bacteria, viruses, spores, molds and fungi, and oxidize many types of undesirable organic and inorganic contaminants in potable waters and wastewaters. It is the choice disinfectant of many drinking water facilities in the U.S and throughout Europe and Asia because of its capability of inactivating microorganisms, including Cryptosporodium and Giardia cysts that are resistant to other types of disinfectants. Recently, ozone has been accepted by the Food and Drug Administration as a disinfectant of food contaminants. Ozone has also been used to inactivate many forms of microorganism in hospital rooms and in brewery cellars. In recent years, ozone has made inroads into commercial laundries and several hospitals in the USA, and is used to disinfect and clean bed linens and towels that may be infected with microorganisms.

### SUMMARY OF INVENTION

The invention is directed to an automated system for controlling ozonation devices and the delivery, in a controlled manner, of ozone for decontaminating enclosed spaces, such as rooms and buildings, to destroy microorganisms and chemicals that contaminate the space or are used as biological or chemical warfare agents. Air with ozone in the gas form is used to completely fill the entire space, including the vents, to inactivate bacteria, viruses, spores, and cysts, and to break down biological and chemical toxins with minimal damage to the contents of the buildings. Ozone decomposes to oxygen in air, and therefore it would not require any cleanup after the decontamination cycle is complete.

Ozone gas, when compared with other decontamination techniques, is particularly effective for decontamination because it is capable of diffusing into crevices and difficult-to-reach areas in buildings. It leaves no residue on treated surfaces. The decontaminated space requires no post treatment cleanup because ozone naturally decomposes into oxygen in a matter of hours. Decomposition of ozone can be further accelerated by directing ozone through catalytic destruction units. In addition, ozone is expected to be effective on both biological and chemical agents.

However, due to its instability, ozone cannot be manufactured and distributed from a central production plant. Instead, it must be generated and applied on-site, at its point of use. Ozone generators from small outputs (g/hr) to large outputs (tons/day) have been items of commerce in the USA and throughout Europe and Asia for many decades. However, they do not include the feed-back control systems set forth herein which allow controlled, effective destruction of biological and chemical agents.

The system includes ozone generators such as set forth in U.S. Ser. No. 09/793,795 filed Feb. 23, 2001, U.S. Pat. No. 6,279,589 issued Aug. 28, 2001, U.S. Ser. No. 09/844,215 filed Apr. 27, 2001, and U.S. Ser. No. 09/977,469 filed Oct. 15, 2001, incorporated herein by reference, covering ozone generators, as well auxiliary devices, which include ozone destruction systems, computer controls, including hardware and software systems, and fully automated high and low-pressure decontamination systems using ozone. The areas of applications include decontamination of containers, trailers, and industrial and food containers; washing of fresh produce, shellfish and other food items for disinfection and extending their shelf life; and full water reclamation systems that are used to treat agricultural, industrial, and urban runoff.

Because biological agents vary in hardiness, customized decontamination cycles are required to kill the various different species. The system described herein addresses the complexity of dealing efficiently and effectively with biological materials, for example, bacterial in both the vegetative state as well as the spore form, providing control of numerous variables. Viruses, molds, funguses, and cysts all have individual life cycles and preferred environments for multiplication and survival. Chemical agents have similar varied requirements but are less complex in nature.

### BRIEF DESCRIPTION OF DRAWINGS

FIG. 1 is a schematic drawing showing a decontamination system for delivering ozone to an enclosed space.

FIG. 2 is a schematic drawing showing a decontamination system for delivering ozone to an air handling system feeding several enclosed spaces.

FIG. 3 is a schematic drawing showing components of the decontamination control portion of the system of FIGS. 1 and 2.

### DETAILED DESCRIPTION

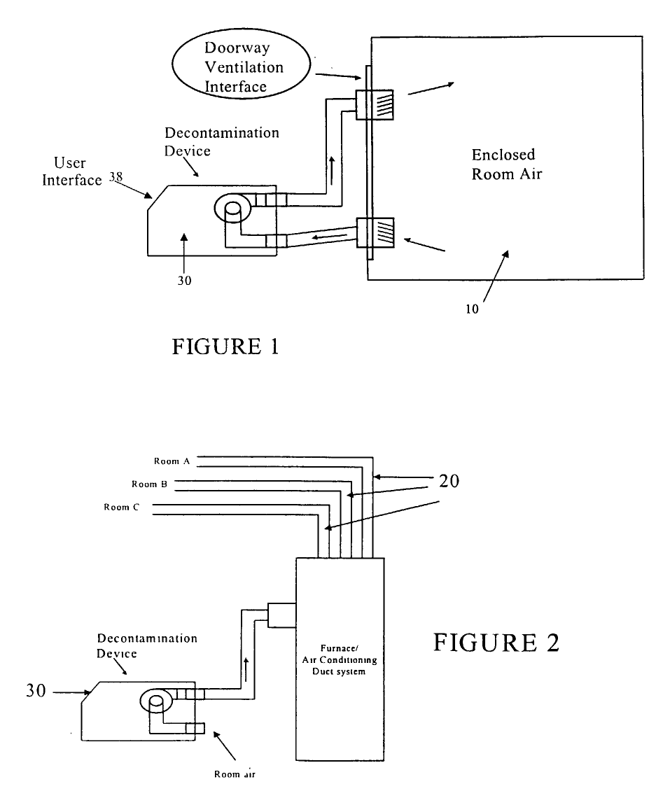

The system described herein is intended to address the many variables and control the automated ozone-generating device under operating conditions suitable for the most effective use of ozone in destruction of biological and chemical agents in enclosed spaces. The system can be used to decontaminate single rooms 10 using ozone gas, as depicted in FIG. 1. Alternatively, FIG. 2 shows an alternative application involving flooding the duct system 20 of a building with ozone gas for complete building decontamination. In such cases, the remediation procedure preferably starts with removal of easily oxidized items, such as rubber, sealing of the affected area(s), using an appropriately sized ozone generating device 30. After the appropriate preprogrammed period of time, the device 30 is automatically turned off, the enclosure is opened and fresh air is allowed to enter the treated space.

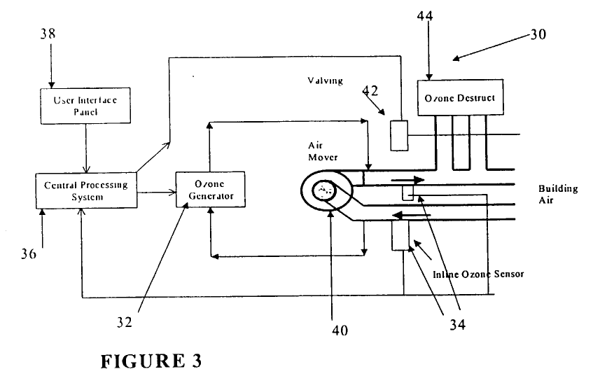

The device 30 can be scaled to meet various demands for different room and building sizes and types. The device incorporates an ozone generator 32, various sensors 34 to monitor ozone concentration, moisture, temperature and other operational variables indicative of controlled conditions and resultant effects, i.e. reduced biological load in influent and effluent air. Process controllers (a central processing unit) 36 will allow adjustment of the rate of ozone generation and other operational variables, (moisture, temperature, etc.) and turning the ozone generator on and off through a feedback mechanism, which can be monitored, controlled and adjusted through a user interface 38. A gas distribution device (air handler) 40 and valving 42 is included. An ozone destruct system 44 can be used when needed. The user interface 38 is preferably provided through a touch-keypad with large buttons, or other suitable control entry means typical for data entry and control systems. FIG. 3 shows the major components required to accomplish a decontamination cycle.

The system is a new assembly using applicant's unique ozone generator applied to distribution, incorporating ozone sensors and conventional process controllers. The described system is the first application of this technology for air handling using high-level ozone measurement sensors. The unit is mobile and capable of being quickly deployed as needed. The chosen interface method allows rapid system setup by individuals clothed to handle hazardous material.

Evaluation of the system described has been conducted by using Bacillus subtilis, a spore forming non-pathogenic bacteria that belong to the genus Bacillus and shares the same physiological characteristics as Bacillus anthracis that causes the infectious anthrax disease. Bacillus subtilis, and Bacillus globigii have been used before as simulants or surrogates of anthrax bacteria in earlier tests involving hydrogen peroxide foam, radiation and even ozone (Masaoka, et al., Applied and Environmental Biotechnology, 1982; Currier, et al., Ozone Science and Engineering, 2001). The system has particular utility in destroying anthrax bacteria. Anthrax is an immediate and the most current public health threat. Aerosolized anthrax bacteria can be present in lethal dosages for body contact or inhalation. The bacteria may be introduced into the ambient air by opened contaminated packages or envelopes, or through the venting systems of buildings. Due to their size, the spores after being introduced into the air primarily settle onto surfaces, such as desks, furniture, clothing, walls, rugs, floors, etc. Delivery of ozone using the described system provides a highly effective means for decontamination of ambient air and the surfaces in rooms, dwellings, offices, buildings, etc. that may have been exposed to anthrax spores or, for that matter, other biological or chemical contaminants.

Although ozone was shown to be effective in gas form in inactivating Bacillus subtilis and Bacillus globigii as well as other biological agents, the information in the literature is sparse, and very little or no data are available on the required ozone concentration, contact time, the ozone demand of different type of surfaces, the rate of inactivation of spores on different surfaces, and the effect of parameters, such as air humidity and temperature on the inactivation rate, which constitute operational variables (can be changed by the operator) and system variable which are different for each situation but are fixed for that particular contaminated site. These types of data are essential for proper sizing of the units for full-scale implementation.

In order to properly control a decontamination system involving either manual or automated control, an algorithm has been developed for the operation of the system to effectively destroy chemical and biological agents with the repeatability required for safe and effective operation. To use ozone as a primary inactivation agent, key environmental factors (system variables) need to be known along with the type of agent be removed.

A general decontamination cycle may have five critical time components, with the summation determining the total decontamination time for the area of contamination.

Total time=(time 1+time 2+time 3+time 4+time 5)

Where:

Time 1—hydration cycle

Time 2—ozone application and hydration cycle

Time 3—ozone application cycle

Time 4—ozone destruct cycle

Time 4—ventilation cycle

Depending on the contaminant and the space being decontaminated, all time components may not be required to remediate a space.

The number of variables involved in decontamination may be infinite. However, it has been determined that by controlling the major contributors to destruction of an agent by ozone, the less significant second and third order variables are small by comparison and can therefore be ignored.

The Primary Variables are:

The nature of the contaminant, either chemical or biological

The cubic volume of the contaminated space

Material compatibility (ozone demand and oxidization resistance)

The temperature of the space

The humidity of the space

The nature of the replacement air supplied to the space, and

System limiting variables (equipment capabilities)

Examples of Secondary Variables are:

Controlling changes in temperature, humidity, ozone output, during a cycle to less than 20% variation

Changes in the contaminant occurring as a result of delivery of ozone during a decontamination cycle.

Contaminant reduction based on oxidation

Variable Interactions

In order to fully describe the complexity of such a system, reference is made to an exemplary biological contaminant, anthrax bacillus. It can be present in both vegetative and spore form as part of its natural life cycle. Under the vegetative form it is easily destroyed by ozone gas, as shown by published reports. This information can be measured and characterized by testing with surrogates. Once the contact time, ozone concentration (CT), temperature and humidity in the contaminate space are determined, ozone gas remediation can be used to effectively decontaminate the space using the equipment described above.

The system utilizes data provided from several sensors 34 in combination in the decontamination algorithm set forth below to self-correct for changes as a result of external influences once a cycle is started. By using a computerized control system, an information feed back loop and this algorithm, a reliable controlled operation can be expected when compared to manual or simply automated cycles.

Within a closed system, the primary variable effecting biological and chemical reactions which constitute the decontamination process are the pressure within the system, the volume being treated the characteristics of the contaminant and the time of ozone exposure. In the case of an enclosed building or room, the pressure is assumed to be a constant. The gas is assumed to be a majority of air, so the primary variables to be controlled are the temperature and exposure times.

Time Component 1—Hydration Cycle

For a spore form bio-contaminant, a hydration cycle is required to cause the spore to open up making it susceptible to ozone. Each spore form contaminants will have different requirements for hydration times at given temperatures, and are characterized on an ozone/humidity resistance scale. Practical hydration is limited to about a 30 to 95% range due to temperature variations within the room, the ceiling to floor distance and condensation, the capacity of the humidifier, and the absorption of materials within the enclosed space. Condensation will primarily be a function of temperature and humidity

T₁=C₁Q/H

T₁=time required for moisture addition

C₁=Concentration of humidity in the room

Q=Controlled Space Volume

H=Total humidity required in the room in % 30%<H<90%

Air saturation is determined by the humidity/temperature/pressure steam tables available from any thermodynamics reference.

Time Component 2—Ozone+Humidity Application

Once the room has come up to the required hydration for effective kill of a selected microorganism, at the temperature of the space, the ozone decontamination cycle is started. During this cycle, the ozone concentration is kept high enough to deactivate the microorganism or chemical without detrimental effects on the interior materials. In addition to ozone, moisture is also added to maintain humidity levels, which can vary as a result of the effects of air exchange and continued absorption of water into materials in the enclosed space. Excess ozone is known to have detrimental effects on materials. Plastics will stiffen, rubbers will crack and fabric will loose its color or brilliance.

T₂=C₁Q/HO₃+U

U=Make up Humidity and Ozone required to compensate for air entering the enclosed space

T₂=Time required for ozone+humidity treatment

C₁=Concentration of humidity in the room

Q=Controlled Space Volume

H=Total humidity required in the room in % 30%<H<90%

O₃=Total required ozone concentration level needed in the room to kill microorganism

Time Component 3—Ozone Treatment

After the ozone has done most of its damage to the contaminant, and adequate hydration has been maintained for a sufficient period of time, additional hydration is no longer required. Ozone concentrations of 6 to 1000 ppm are continued until the microorganism is known to be killed by statistical analysis and data collected during trial phase experimentation.

T₃=C/O₃+M

When M falls to 25% of T₂ ozone demand, Disinfections is assumed to be complete.

T₃=Time required for ozone treatment

C=Ozone measured, which is less than or equal to ozone level O₃

O₃=Ozone level required for killing microorganisms

M=Makeup ozone, the amount of input required to maintain a set level in the enclosed space. (decreases as oxidation occurs on internal surfaces of the enclosed space)

Time Component 4—Ozone Destruct Cycle

This time is specifically for destruction of the ozone gas by recirculating the air within the space which may include use of an ozone destruct device 44 to destroy the ozone present in the room. Chemical catalysts can be used to degrade ozone back to oxygen but they tend to foul as dust and particulate accumulate on the media bed. An alternative method is to use UV light at 235 to 255 nm to degrade the ozone in the air recirculation stream. This method is preferred because of reduced fouling and beneficial germicidal effects of UV light.

T₄=EQ/C−N

T₄=Time required to reduce the ozone level to </1 ppm in the enclosed space

E=UV Energy required to convert O₃ to O₂ (using a practical sized fixed UV tube)

C=measured levels at sensor

N=Ozone lost due to makeup air entering the room

Time Component 5—Ventilation

After the ozone in the room falls below 0.1 ppm, the room decontaminated of biological contaminates is safe to enter. However, chemical oxidation by-products may be present in the room air needing ventilation to bring levels to acceptable levels before allowing occupants to enter the confined space.

T₅=Q/F

T₅=Time required for ventilation

Q=Volume of the enclosed space

F=Cubic feet per minute of air exchange based on system blower capability

Total algorithm is as follows:

Tₜₒₜₐₗ=[(C₁Q/H)+(C₁Q/HO₃+U)+(C/O₃+M)+(EQ/C−N)+(Q/F)]

It is evident from the foregoing that there are many additional embodiments of the present invention which, while not expressly described herein, are within the scope of this invention and may suggest themselves to one of ordinary skill in the art. For example, the invention is not limited to anthrax decontamination but is broadly applicable to destruction of numerous bacteria or viruses (smallpox, etc.). It also is not limited to use on biological warfare agents but is suitable for destruction of many naturally existing environmental contaminants, such as mold and mildew, and chemical agents subject to oxidation to render them non-toxic.

It is therefore intended that the invention be limited solely by the appended claims.

We claim:

1. A system for provided controlled quantities of gaseous ozone for a predetermined controlled period of time to an enclosed space to reduce biological or chemical contaminants within that enclosed space to safe or non-existent levels comprising:
a) an enclosed air delivery system for providing a moving air stream, said air stream supplemented with ozone, to the enclosed space, removing air from the enclosed space, adding ozone to the air removed from the enclosed space, and returning the air supplemented with additional ozone to the enclosed space,
b) an ozone generator for delivering controlled quantities of gaseous ozone to the moving air stream in the enclosed air delivery system,
c) sensors located within the enclosed air delivery system or enclosed space for monitoring one or more operational variables, said operational variables comprising ozone concentration, moisture content, temperature and quantity of the biological or chemical contaminant in the moving air stream,
d) a computerized control system programmed to control said operational variables in response to data received from the sensors, said computerized control system also including a user interface for receiving operator input as to one or more system variables, said system variables comprising volume of the enclosed space being treated, characteristics of the biological or chemical contaminant, response of the biological or chemical contaminant to operational variables and treatment times based on the said system variables.

2. The system of claim 1 wherein the sensors are located at least in the air stream entering the enclosed space and the air steam air removed from the enclosed space.

3. The system of claim 1 further including an ozone destruct system.

4. The system of claim 1 wherein the treatment times comprise one or more of 5 time cycles comprising a first time cycle for hydration, a second time cycle for ozone application with hydration, a third time cycle for ozone application without hydration, and fourth time cycle for ozone destruction and a fifth time cycle for enclosed space ventilation, the sum of time for one or more of the first, second, third, fourth and fifth time cycle being the total treatment time.

5. The system of claim 4 wherein the total time is defined by:
Tₜₒₜₐₗ=[(C₁Q/H)+(C₁Q/HO₃+U)+(C/O₃+M)+(EQ/C−N)+(Q/F)]
wherein:
C₁=Concentration of humidity in the room,
Q=Controlled Space Volume,
H=Total humidity required in the room in % where 30%<H<90%,
U=Make up Humidity and Ozone required to compensate for air entering the enclosed space,
O₃=Total required ozone concentration level needed in the room to kill microorganism,
C=Measured Ozone level which is less than or equal to ozone level O₃
M=Makeup ozone,
E=UV Energy required to convert O₃ to O₂ (using a practical sized fixed UV tube),
N=Ozone lost due to makeup air entering the room, and
F=Cubic feet per minute of air exchange based on system blower capability.
